(19) United States
(12) Patent Application Publication
(10) Pub. No.: US 2003/0031588 A1
(43) Pub. Date: Feb. 13, 2003

(54) Device for enriching air with an air treatment agent, especially for the disinfection of air, and/or perfuming of air and/or for odor masking

(76) Inventor: Jorg Peter Schur, Wegberg-Dalheim (DE)

Correspondence Address: DANN DORFMAN HERRELL & SKILLMAN, SUITE 720, 1601 MARKET STREET, PHILADELPHIA, PA, 19103-2307, US

(21) Appl. No.: 10/172,340

(22) Filed: Jun. 13, 2002

Related U.S. Application Data
(60) Provisional application No. 60/318,287, filed on Sep. 10, 2001.

(30) Foreign Application Priority Data
Jun. 13, 2001 (DE) 10128563.9
Aug. 25, 2001 (DE) 10141734.9

(51) Int. Cl. A61L 9/00; A62B 7/08

(52) U.S. Cl. 422/28; 422/5; 422/32; 422/124; 422/125; 422/306; 422/307

(57) ABSTRACT

A device for enriching air with an air treatment agent comprises a storage vessel for receiving liquid air treatment agent. An evaporating means for heating the air treatment agent is connected with the storage vessel through a pipe. A dosing means provided between said storage vessel and said evaporating means produces a quantity-restricted supply of air treatment agent to said evaporating means. The evaporating means is connected with a mixing vessel in which the vaporized air treatment agent is mixed with air, and the mixture of vaporized air treatment agent and air is exhausted through an outlet into the space to be treated with air treatment agent.

The present invention relates to a device for enriching air with an air treatment agent, especially for the disinfection or perfuming of air, and for odor masking.

The treatment of air is required, for example, in living spaces (e.g., of allergic persons), office buildings, means of transportation, hygienic areas and public health institutions. For the treatment of air, evaporators are known in which an air treatment agent is evaporated with the aid of an evaporating means. When the air treatment agent is evaporated in this manner, the air is enriched with treatment agents to a relatively high extent so that the treatment agent will precipitate in the space to be treated. Even the pulsing of an evaporator which operates by the input of heat cannot avoid the precipitation of the air treatment agent; the precipitation will only be intermittent. The precipitation of air treatment agent on cold objects, such as windows and the like, annoys the user and further leads to a higher tendency to soiling of these objects since dust, for example, is attracted by the precipitate. Further, precipitation on wooden furniture and the like can result in damage to the furniture.

Further, pneumatic spray systems for the spraying of air treatment agents are known. This involves fine atomization of the air treatment agent. The use of pneumatic spray systems does not avoid the occurrence of precipitations of the air treatment agent either.

Another field of application for air treatment agents is, for example, the introduction of sterilizing agents during the cooling of bakery products after the baking process. It must be avoided that mold germs will settle on the surface of the bakery products prior to packaging. Since precipitation of the air treatment agent is not acceptable in this field either, expensive air filtering systems of different kinds are employed. This involves the problem that the mold germs can deposit in the air filtering system and act as molding sources for the air filter itself. Consequently, the filters must be replaced frequently and cleaned very thoroughly.

Air treatment is further necessary in the storing of cheese after maturation since undesirable molding then occurs on the surface of the cheese form the mold germs present in the air. To avoid this, cheese is coated, for example, with a coating agent which contains an antibiotic. Due to diffusions, the antibiotic penetrates into the external portion of the cheese. This causes antibiotic to be unintentionally supplied to the human body when the cheese is eaten. The use of filtering systems in cheese production has the same disadvantage as in the production of bakery products.

It has been the object of the invention to provide a device for enriching air with an air treatment agent in which precipitation of the air treatment agent is avoided.

According to the invention, this object is achieved by the features of claims 1 and 10.

In a first preferred embodiment, the device according to the invention comprises a storage vessel for receiving liquid air treatment agent, and an evaporating means for heating the air treatment agent, connected with the storage vessel, for example, through a pipe or hose. According to the invention, a dosing means is provided between the storage vessel and the evaporating means, i.e., for example, within said hose or pipe. The dosing means ensures a rate-limited supply of liquid air treatment agent to the evaporating means. According to the invention, a mixing vessel in which the evaporated air treatment agent is mixed with air is connected with the evaporating means. For discharging the mixture of air and vaporized air treatment agent, the mixing vessel comprises an outlet. According to the invention, air is supplied to the mixing vessel, for example, through inlet openings, at such a high rate, in relation to the supplied low amount of liquid air treatment agent, that it is possible to supply to the space to be treated an amount of air treatment agent per hour and cubic meter of air which is between 0.1 and 0.00001 ml, preferably between 0.01 and 0.001 ml. Due to this low amount of air treatment agent supplied to the space to be treated, a precipitate of the air treatment agent cannot be detected in the space. Therefore, an annoying precipitate on cool windows or the like does not occur. Thus, the device according to the invention is especially suitable for living spaces, waiting rooms and the like.

In the mixture of vaporized air treatment agent and air leaving the outlet of the mixing vessel, the proportion of air treatment agent is preferably lower than 100 ppb (parts per billion), more preferably lower than 10 ppb.

Preferably, a means for generating a current of air, such as a fan, is assigned to the mixing vessel for mixing the vaporized air treatment agent with air. The fan sucks or blows air into the mixing vessel through the air inlet openings provided in the mixing vessel. Further, the means for generating a current of air serves for ejecting or exhausting the mixture of air and vaporized air treatment agent out of the outlet.

Preferably, a quantity of from 0.01 ml per cubic meter and per hour to 0.005 ml per cubic meter and per hour of air treatment agent is supplied to the space to be treated. Thus, for example, for a space of 50 cubic meters of air, a quantity of from 0.5 ml per hour to 0.25 ml per hour is supplied from the storage vessel to the evaporating means and evaporated therein.

Especially in order to avoid dragging of droplets of air treatment agent by the current of air, the delivery rating of the means for feeding the current of air must not be too high. On the other hand, a relatively high feed volume is required in order to achieve an extremely low proportion of air treatment agent per cubic meter of air. For a quantity of air treatment agents to be evaporated of from 0.25 to 0.5 ml per hour, the delivered volume flow rate of air is preferably from 25 to 35 m³ per hour. Thus, the ratio of the delivered quantity of air and the quantity of air treatment agent supplied to the evaporating means is within a range of from 140/1 to 50/1, preferably from 100/1 to 70/1.

In order to avoid reduced pressure in the mixing vessel, said at least one air inlet opening of the mixing vessel has a greater cross-sectional area than said at least one outlet. When several outlets or several inlet openings are present, the sum of the cross-sectional areas of the outlets is respectively smaller than the sum of the cross-sectional areas of the air inlet openings.

In a preferred embodiment of the invention, the air treatment agent is continuously supplied to the evaporating means. To ensure this, the dosing means preferably has an outlet through which the air treatment agent is discharged towards the evaporating means and which has a cross-sectional area of smaller than 0.078 cm², especially smaller than 0.000314 cm². When the outlet is round, this corresponds to a diameter of 0.1 mm or 0.2 mm. Preferably, the outlet aperture is predetermined and non-variable. Varying the diameter of the outlet can be effected, for example, by changing the corresponding disk or the like in which the outlet is provided. Also, it is possible to provide a disk or the like having a relatively large outlet aperture and to reduce the quantity of air treatment agent supplied to the evaporating means by providing disks having smaller outlet apertures upstream or downstream of the disk having the large air outlet apertures. Thus, for example, insertion slots for inserting such disks can be provided in a pipe or hose arranged between the storage vessel and the evaporating means.

Preferably, the storage vessel is arranged relative to the evaporating means to have these two parts of the device at different levels, the storage vessel being arranged at a higher level. This produces a slope in the fluid communication, such as the pipe or hose between the storage vessel and the evaporating means. This enables a continuous supply of air treatment agent to the evaporating means without having to provide a pump or other conveying means. Thus, the only energy consumers of the device according to the invention are the evaporating means and the fan.

Since only very low amounts of air treatment agent must be evaporated and air treatment agents generally are relatively volatile substances, the evaporating means preferably has a temperature of from 40 to 70° C.

Further, it is possible to provide a peristaltic pump or some other suitable conveying means for conveying the air treatment agent instead of a dosing means having a correspondingly adapted small outlet aperture. Preferably, the air treatment agent is conveyed continuously. The amount of air treatment agent is dependent, on the one hand, on the feed volume of the fan and, on the other hand, on the capacity of the space to be treated. Instead of continuous feeding, it is also possible to supply the air treatment agent intermittently to the evaporating means. This has the advantage that conventional inexpensive peristaltic pumps can be employed.

Preferably, the storage vessel comprises a pressure compensation means so that there is always ambient pressure rather than reduced pressure in the storage vessel. Reduced pressure would influence the amount of air treatment agent supplied to the evaporating means. The storage vessel is preferably sealed with a cover or the like. The cover, which can be removed for filling the storage vessel, preferably comprises a particle filter. The particle filter is designed in such a way that air can flow into the storage vessel for pressure compensation while prevent particles, such as dust, from entering the storage vessel. Such particles could obstruct the dosing means or affect the flow rate. The pressure compensation means, which is preferably an aperture provided with a particle filter, is preferably provided in the cover. However, it may also be arranged in a different place on the storage vessel.

A second preferred embodiment of the invention for enriching air with an air treatment agent also comprises a storage vessel for receiving a liquid air treatment agent, and an evaporating means, connected with the storage vessel, for heating the air treatment agent. This preferred embodiment of the invention has an inclined evaporation surface of the evaporating means. The air treatment agent flows over this inclined evaporation surface. According to the invention, a recirculating means which recirculates the non-evaporated air treatment agent into the storage vessel is connected with the evaporating means. Thus, a thin film of air treatment agent preferably forms on the evaporation surface of the evaporating means so that a uniform evaporation of the air treatment agent occurs throughout the surface of the evaporation surface.

By providing a recirculating means which collects excess air treatment agent to recirculate it into the storage vessel, it is achieved that the amount of air treatment agent supplied to the evaporating means needs not to be determined too exactly. Rather, the amount of air treatment agent which is evaporated and supplied to the air to be treated essentially depends on the temperature of the evaporation surface of the evaporating means. Thus, the amount of air treatment agent supplied to the air to be treated can be adjusted simply by controlling the temperature of the evaporation surface. Further, the device according to the invention has the advantage that no depositions are formed on the evaporation surface due to the fact that the air treatment agent preferably flows continuously over the evaporation surface. Depositions can adversely affect the efficiency of the evaporating means and require that the evaporating means is regularly cleaned. In the device according to the invention, this cleaning is not required, or at most so only at very long intervals.

The inclination of the evaporation surface, which is preferably arranged in a plane, is preferably from 100 to 300 relative to a horizontal plane. Particularly preferred is an inclination of from 15° to 25°.

In order to ensure a directed flow of the air treatment agent over the evaporation surface, the evaporation surface is preferably provided with conveying grooves running in the direction of flow of the air treatment agent.

Preferably, the second embodiment of the invention comprises a mixing vessel designed in accordance with the above described first embodiment of the invention. Preferably, a means for generating a current of air, such as a fan, is arranged within the mixing vessel. It is particularly preferred to arrange the fan below the evaporating means so that air flows alongside the evaporating means, thus being enriched with evaporated air treatment agent. Arranging the fan below the evaporating means has the advantage that the evaporation surface is not cooled down too much by the air current generated by the fan, so that the desired evaporation rate is retained.

The second embodiment of the invention with an inclined evaporation surface can be further developed advantageously in accordance with the above described first embodiment.

For the use of the device according to the invention for the sterilization of air, an antimicrobial composition (X) is preferably used as the air treatment agent. Preferably, the antimicrobial composition contains one, two or more GRAS (generally recognized as safe) flavoring agents or their derivatives. Preferred antimicrobial compositions comprising two or more GRAS flavoring agents have been described in Published International Application No. WO 01/03747 on pages 5 to 14, herein incorporated by reference.

For the odor neutralization of air, there is preferably used an odor-masking composition (Y) which contains at least one odor-masking component (A) selected from terpenes, corn starch, manganese salts, essential oils and polyvinyl pyrrolidone, as referenced in German Patent Application No. DE 101 00 595. In addition, antimicrobial compositions comprising only one GRAS flavoring agent, as specified below, may also be employed, such as compositions containing only one GRAS alcohol such as propylene glycol or benzene alcohol.

The preferred compound of the odor-masking component (A) is polyvinyl pyrrolidone (polyvidone; poly(2-oxo-1-pyrrolidinyl) ethylene; poly(1-vinyl-2-pyrrolidone); hereinafter sometimes briefly referred to as “PVP”), especially PVP having a molecular weight of from 10,000 to 60,000 g/mol, preferably from 30,000 to 50,000 g/mol. Particularly preferred is PVP having a molecular weight of about 40,000 g/mol, i.e., this is a PVP having a certain degree of cross-linking (i.e., a viscosity of from 15 to 25, preferably about 2 mPa·s at 20% by weight in water). The proportion of odor-masking component (A) in the odor-masking composition is preferably within a range of from 0.001 to 50% by weight, more preferably from 0.1 to 10% by weight.

According to embodiment (2), the odor-masking composition (Y) can contain an additional functional flavor component (B), which preferably contains one or more of the following substances:

hexyl butyrate, octyl acetate, isobutyl isobutyrate, cis-3-hexene-1-yl acetate, γ-decalatone, ethyl caproate, butyl acetate, ethyl benzoate, ethyl butyrate, hexyl acetate, methyl caproate, phenylethyl alcohol, citronellol, undecyl aldehyde, benzylphenyl acetate, cinnamyl alcohol, eugenol, benzyl acetate, linalool, cis-jasmone, acetylmethyl anthranilat, cis-3-hexene-1-ol, cis-3-hexene-1-yl salicylate, methyl benzoate, methyl salicylate, geranyl acetate, cis-3-hexene-1-yl acetate, Litsea cubeba, orange oil, phenylpropyl alcohol and phenylethyl acetate.

Preferably, the proportion of the functional flavor component (B) is from 0.001 to 20% by weight, preferably from 0.1 to 5% by weight, of the odor-masking composition.

According to the present invention, the odor-masking composition (Y) may further contain a flavor component (C) which is selected from essential oils, flavors and fragrances. The proportion of the flavor component (C) in the odor-masking composition is from 0.01 to 95% by weight, preferably from 0.1 to 80% by weight.

In a particularly preferred embodiment, the flavor component (C) contains antimicrobial substances; preferably, it contains at least one GRAS (generally recognized as safe) flavoring agent. Of these, particularly preferred are those flavor components (C) which contain aromatic GRAS flavor alcohol (such as benzyl alcohol, cinnamyl alcohol, α-methylbenzyl alcohol and anisalcohol, benzyl alcohol being preferred) or a GRAS polyphenol compound, or those containing at least two GRAS flavoring agents. It has been found that especially those flavor components (C) which contain

one or more GRAS flavor alcohols or their derivatives; and

one or more flavoring agents selected from

(b1) polyphenol compounds; and

(b2) GRAS flavor acids or their derivatives;

are particularly preferred.

The mentioned GRAS flavor alcohols of component (A) and the components (B) to (H) defined hereinbelow are recognized by the FDA authority as commercially safe for use in foods (GRAS=generally recognized as safe in food). The mentioned GRAS flavor alcohols and also the other GRAS flavoring agents defined below are the compounds mentioned in the FEMA/FDA GRAS Flavour Substances Lists GRAS 3-15 No. 2001-3905 (as of 2000). This list contains natural and synthetic flavoring agents approved by the American public health authority, FDA, for use in food-stuffs: FDA Regulation 21 CFR 172.515 (Synthetic Flavoring Substances and Adjuvants) and FDA Regulation 21 CFR 182.20 (Natural Flavoring Substances and Adjuvants).

The flavor component (C) can contain

from 0.1 to 99.9% by weight, preferably from 0.5 to 99% by weight, of component (A);

from 0 to 25% by weight, preferably from 0.01 to 10% by weight, of component (b1); and/or

from 0 to 70% by weight, preferably from 0.01 to 30% by weight, of component (b2).

According to the invention, component (A) may contain one or more GRAS flavor alcohols. It is preferred according to the invention to use two or three GRAS flavor alcohols. In detail, the following GRAS flavor alcohols may be employed, for example: benzyl alcohol, acetoin (acetylmethylcarbinol), ethyl alcohol (ethanol), propyl alcohol (1-propanol), iso-propyl alcohol (2-propanol, isopropanol), propylene glycol, glycerol, n-butyl alcohol (n-propyl carbinol), iso-butyl alcohol (2-methyl-1-propanol), hexyl alcohol (hexanol), L-menthol, octyl alcohol (n-octanol), cinnamyl alcohol (3-phenyl-2-propene-1-ol), α-methylbenzyl alcohol (1-phenylethanol), heptyl alcohol (heptanol), n-amyl alcohol (1-pentanol), iso-amyl alcohol (3-methyl-1-butanol), anisalcohol (4-methoxybenzyl alcohol, p-anisalcohol), citronellol, n-decyl alcohol (n-decanol), geraniol, β-γ-hexenol (3-hexenol), lauryl alcohol (dodecanol), linalool, nerolidol, nonadienol (2,6-nonadiene-1-ol), nonyl alcohol (nonanol-1), rhodinol, terpineol, borneol, clineol (eucalyptol), anisole, cuminyl alcohol (cuminol), 10-undecene-1-ol, 1-hexadecanol. As said derivatives, both natural and synthetic (naturally occurring or not) derivatives can be employed. Suitable derivatives include, for example, the esters, ethers and carbonates of the above mentioned GRAS flavor alcohols. Particularly preferred GRAS flavor alcohols are benzyl alcohol, 1-propanol, glycerol, propylene glycol, n-butyl alcohol, citronellol, hexanol, linalool, acetoin and their derivatives.

As component (b1), the following polyphenols may be employed: catechol, resorcinol, hydroquinone, phloroglucinol, pyrogallol, cyclohexane, usnic acid, acylpolyphenols, lignins, anthocyans, flavones, catechols, gallic acid derivatives (e.g., tannins, gallotannin, tannic acids, gallotannic acids), including derivatives of the above-mentioned compounds, such as (2,5-dihydroxyphenyl)carboxylic and (2,5-dihydroxyphenyl)alkylenecarboxylic substitutions, salts, esters, amides; caffeic acid and its esters and amides, flavonoids (e.g., flavone, flavonol, isoflavone, gossypetin, myricetin, robinetin, apigenin, morin, taxifolin, eriodictyol, naringin, rutin, hesperidin, troxerutin, chrysin, tangeritin, luteolin, catechols, quercetin, fisetin, kaempferol, galangin, rotenoids, aurones, flavonols, flavonediols), extracts, e.g., form Camellia, Primula. Further, their possible derivatives, e.g., salts, acids, esters, oxides and ethers, may also be used. A particularly preferred polyphenol is tannin (a GRAS compound).

As component (b2), the following GRAS acids may be used, for example: acetic acid, aconitic acid, adipic acid, formic acid, malic acid (1-hydroxysuccinic acid), capronic acid, hydrocinnamic acid (3-phenyl-1-propionic acid), pelargonic acid (nonanoic acid), lactic acid (2-hydroxypropionic acid), phenoxyacetic acid (glycolic acid phenyl ether),phenylacetic acid (α-toluenic acid), valeric acid (pentanoic acid), iso-valeric acid (3-methylbutyric acid), cinnamic acid (3-phenylpropenoic acid), citric acid, mandelic acid (hydroxyphenylacetic acid), tartaric acid (2,3-dihydroxybutanedioic acid; 2,3-dihydroxysuccinic acid), fumaric acid, tannic acid and their derivatives.

Suitable derivatives of the mentioned acids according to the present invention are esters (e.g., C₁₋₆-alkyl esters and benzyl esters), amides (including N-substituted amides) and salts (alkali, alkaline earth and ammonium salts). According to the present invention, the term “derivatives” also encompasses modifications of the side-chain hydroxy functions (e.g., acyl and alkyl derivatives) and modifications of the double bonds (e.g., the perhydrogenated and hydroxylated derivatives of the mentioned acids).

The mixing ratio of component (A) to component (B) is preferably between 10,000:1 and 1:10,000, more preferably between 1000:1 and 1:1000, and even more preferably between 100:1 and 1:100.

In a preferred embodiment of the process according to the invention, the flavor component (C¹) or the antimicrobial composition (X) contains:

(a1) benzyl alcohol as a necessary component; and optionally

(a2) one or more further GRAS flavor alcohols or their derivatives; and

(b1) one or more polyphenol compounds; and/or

(b2) one or more GRAS acids or their derivatives.

Suitable amounts of components (al), (a2), (b1) and (b2) are:

from 0.1 to 99% by weight, preferably from 0.1 to 75% by weight, of benzyl alcohol;

from 0 to 99.8% by weight, preferably from 0.01 to 99% by weight, of component (a2);

from 0 to 25% by weight, preferably from 0.01 to 10% by weight, of component (b1); and/or

from 0 to 70% by weight, preferably from 0.91 to 30% by weight, of component (b2).

The flavor component (C¹) or the antimicrobial composition (X) may further contain the following components (C) to (H), which are also flavoring agents recognized as GRAS (generally recognized as safe in food) in the FEMA/FDA GRAS Flavour Substances List 3-15 No. 2001-3905 (as of 2000).

As component (C), the following phenol compounds may be employed: thymol, methyleugenol, acetyleugenol, safrol, eugenol, isoeugenol, anethole, phenol, methylchavicol (estragol; 3-(4-methoxyphenyl)-1-propene), carbacrol, α-bisabolol, formesol, anisole (methoxybenzene), propenylguaethol (5-propenyl-2-ethoxyphenol) and their derivatives.

As GRAS esters component (D)), allicin and the following acetates may be used: iso-amyl acetate (3-methyl-1-butyl acetate), benzyl acetate, benzylphenyl acetate, n-butyl acetate, cinnamyl acetate (3-phenylpropenyl acetate), citronellyl acetate, ethyl acetate (acetic ester), eugenol acetate (acetyleugenol), geranyl acetate hexyl acetate (hexanyl ethanoate), hydrocinnamyl acetate (3-phenyl-propyl acetate), linalyl acetate, octyl acetate, phenylethyl acetate, terpinyl acetate, triacetin (glyceryl triacetate), potassium acetate, sodium acetate, calcium acetate. Further suitable esters are the ester derivatives of the above defined acids (component (b2)).

As terpenes (component (E)), there may be used, for example, camphor, limonene and β-caryophyllene.

The acetals (component (F)) which can be used include, e g., acetal, acetaldehyde dibutyl acetal, acetaldehyde dipropyl acetal, acetaldehyde phenethyl propyl acetal, cinnamic aldehyde ethylene glycol acetal, decanal dimethyl acetal, heptanal dimethyl acetal, heptanal glyceryl acetal and benzaldehyde propylene glycol acetal.

As aldehydes (component (G)), there may be used, e.g., acetaldehyde, anisaldehyde, benzaldehyde, iso-butyl aldehyde (methyl-1-propanal), citral, citronellal, n-caprylic aldehyde (n-decanal), ethylvanillin, furfural, heliotropin (piperonal), heptyl aldehyde (heptanal), hexyl aldehyde (hexanal), 2-hexenal (β-propyl-acrolein), hydrocinnamic aldehyde (3-phenyl-1-propanal), lauryl aldehyde (dodecanal), nonyl aldehyde (n-nonanal), octyl aldehyde (n-octanal), phenylacetaldehyde (1-oxo-2-phenylethane), propionaldehyde (propanal), vanillin, cinnamic aldehyde (3-phenylpropenal), perillaldehyde and cuminaldehyde).

The following essential oils and/or alcoholic or glycolic extracts or extracts obtained by CO₂ high-pressure processes from the mentioned plants (component (H)) can also be employed according to the invention:

(h1) oils or extracts having a high content of alcohols: melissa, coriander, cardamon, eucalyptus;

(h2) oils or extracts having a high content of aldehydes: Eucalyptus citriodora, cinnamon, lemon, lemon grass, melissa, citronella, lime, orange;

(h3) oils or extracts having a high content of phenols: origanum, thyme, rosemary, orange, clove, fennel, camphor, mandarin, anise, cascarilla, estragon and pimento;

(h4) oils or extracts having a high content of acetates: lavender;

(h5) oils or extracts having a high content of esters: mustard, onion, garlic;

(h6) oils or extracts having a high content of terpenes: pepper, bitter orange, caraway, dill, lemon, peppermint, nutmeg.

The proportion of components (C) to (H) in the flavor component (C) or (C¹) and in the antimicrobial composition (X) is preferably smaller than or equal to 25% by weight, more preferably within a range of from 0.001 to 9% by weight. Preferred among the further GRAS flavoring agents are the phenols (C) and essential oils (H).

Particularly preferred according to the present invention is flavor component (C) or (C¹) in which the antimicrobially active component exclusively consists of GRAS flavoring agents, i.e., which does not contain any “derivatives” of the GRAS flavoring agents. As an example of such a composition, there may be mentioned a mixture of benzyl alcohol, one or two of the above mentioned GRAS flavor alcohols (a2) and tannin. Such a mixture preferably contains from 0.1 to 99.9% by weight, more preferably from 0.1 to 20% by weight, of benzyl alcohol, and from 0.01 to 10% by weight of tannin. Another example of a preferred composition is a mixture of 2 alcohols, a polyphenol (especially tannin) and an essential oil (especially a phenolic essential oil, component (h3)).

In addition to components (A) to (C), further compounds (D), such as alcohols (d1), emulsifiers (d2), stabilizers (d3), antioxidants (d4), preservatives (d5), solvents (d6), carriers (d7) etc., may additionally be employed. The proportion of components (D) in the odor-masking composition may be up to 99%0 by weight, is preferably smaller than 50% by weight, and is more preferably within a range of from 0.1 to 20% by weight.

According to the invention, the alcohols (d1) are monohydric or polyhydric alcohols having from 2 to 10 carbon atoms, preferably having from 2 to 7 carbon atoms, wherein the GRAS alcohols (a) are excluded. Preferably, the GRAS flavor alcohols (A) and further alcohols (d1) are employed in such amounts that their mixing ratio is between 1000:1 and 1:1000, especially between 11:1 and 1:100, and more preferably between 10:1 and 1:10.

The carriers D7 are preferably polymeric compounds, such as polyethylene glycol, polypropylene glycol etc.

In certain applications, for example, when the odor-masking composition is contacted with foodstuffs or employed in rooms where people live, it may be appropriate to employ systems which are free from ethanol and isopropanol, or free from harmful doses of ethanol or isopropanol, since these substances can be absorbed by foods, for example, and may also be inhaled by the people in the treated rooms. In addition when such compounds are used, there may be danger of explosion.

In particular, the above described device may also be used in connection with a refrigerating means, such as a household refrigerator. It is thus possible to introduce a small amount of air treatment agent, especially air sterilizing agent or odor-masking air treatment agent, into the interior or the refrigerator or the refrigerating means.

Another preferred use of the device according to the invention is in connection with a computer. Due to the heat occurring within a computer and the dust accumulating in the air, the fan emits a large number of germs into the ambient air. Therefore, the device according to the invention is preferably arranged in the zone of the fan of a computer. Now, the current of air produced by the computer fan can be utilized for carrying the air treatment agent. Thus, the germ-bearing exhaust air from the computer is immediately treated with an air treatment agent. Further, the device according to the invention can be arranged in the air suction zone of the fan so that the formation of germs is suppressed from the beginning.

Both in the refrigerating means and in the computer, it is possible to provide the device according to the invention either on the outside of the housing or in the interior of the refrigerating means or computer. It is further possible to use the heat sources of the refrigerator or computer as an evaporating means. Especially in computers, an additional evaporating means may optionally be dispensed with altogether.

In the following, the invention is further illustrated by preferred embodiments with reference to the accompanying drawings.

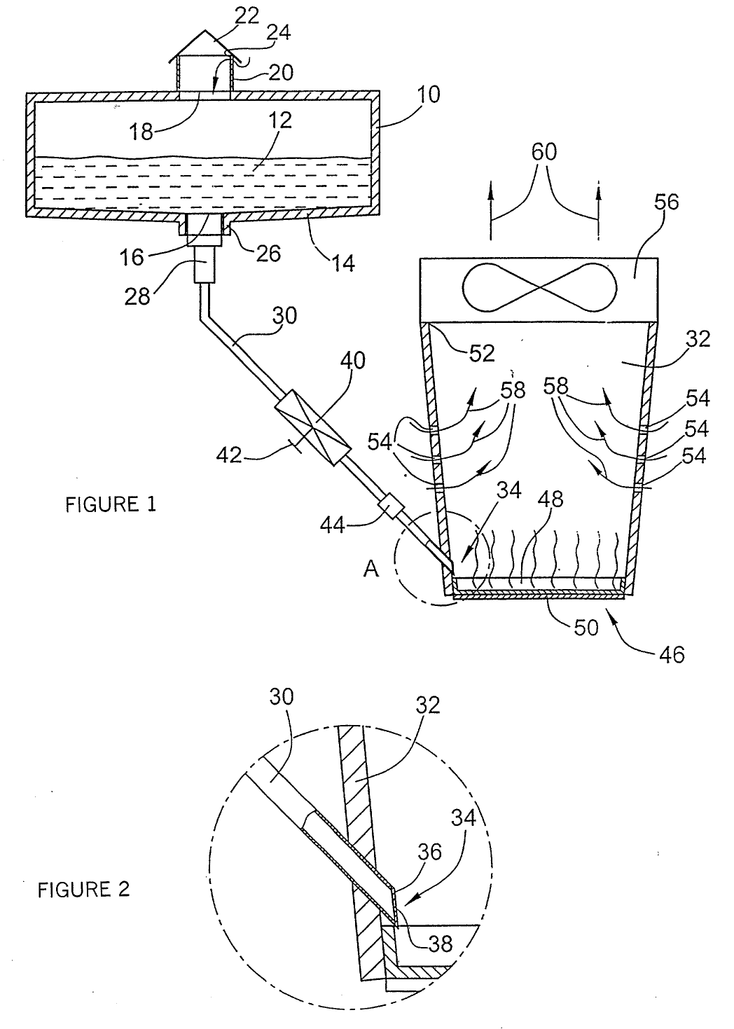

FIG. 1 shows a schematic side view of a preferred embodiment of the invention;

FIG. 2 shows an enlarged view of segment A of FIG. 1;

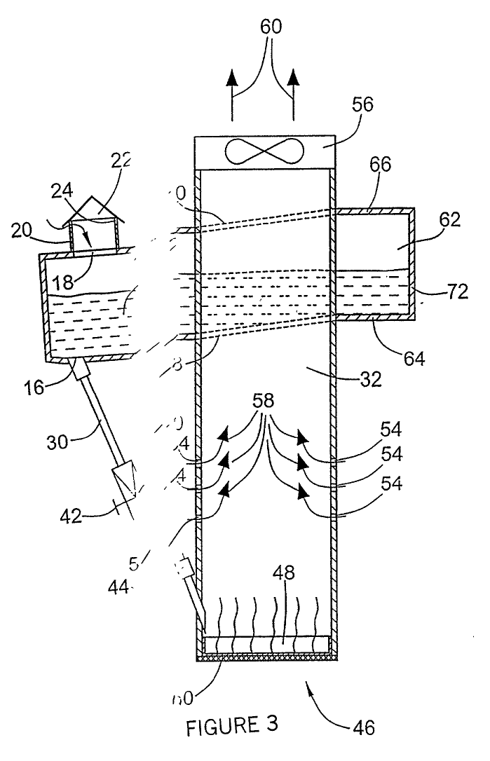

FIG. 3 shows a schematic side view of a further preferred embodiment of the invention;

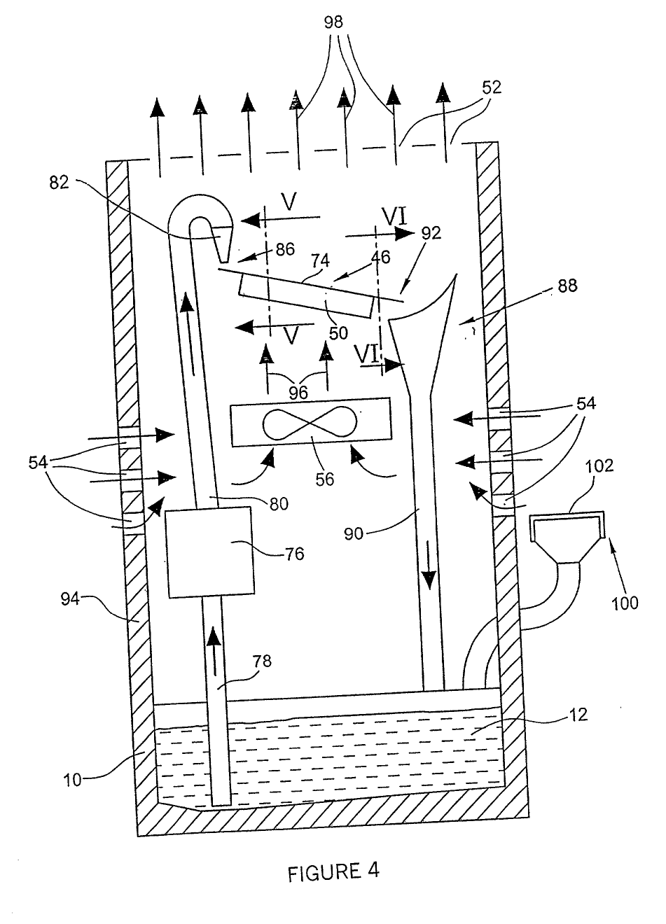

FIG. 4 shows a schematic side view of a further preferred embodiment of the invention;

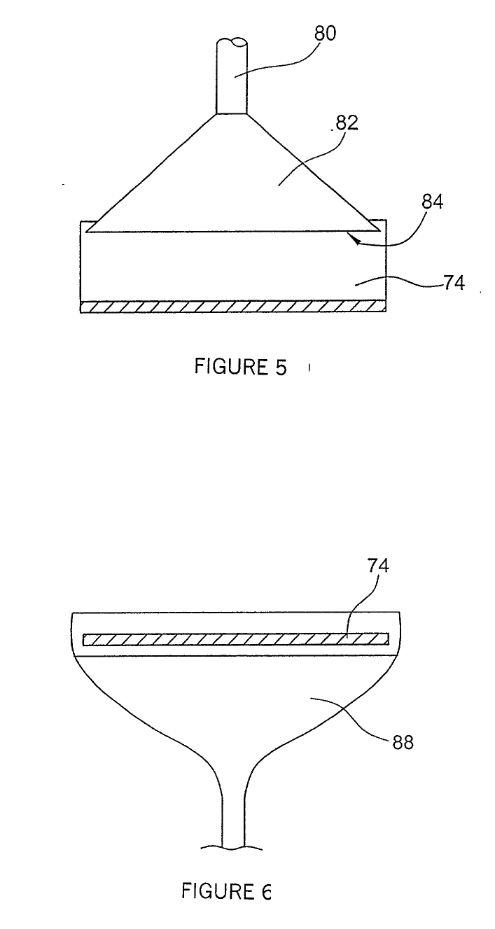

FIG. 5 shows a sectional view taken along V-V in FIG. 4;

FIG. 6 shows a schematic sectional view taken along VI-VI in FIG. 4;

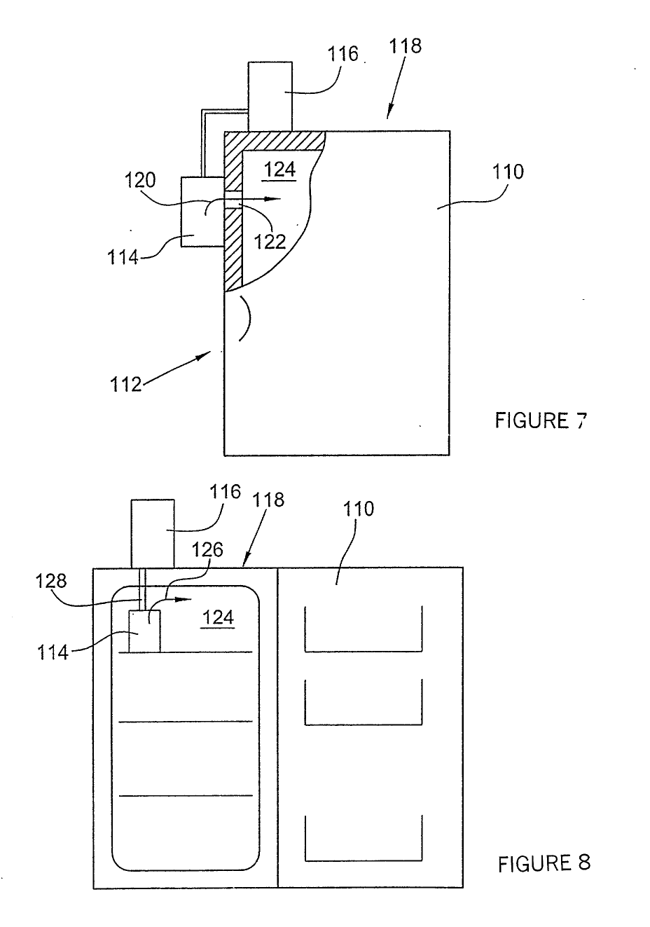

FIG. 7 shows a schematic view of a household refrigerator with a device for enriching air provided in the exterior of the refrigerator;

FIG. 8 shows a schematic view of a household refrigerator with a device for enriching air provided in the interior of the refrigerator; and

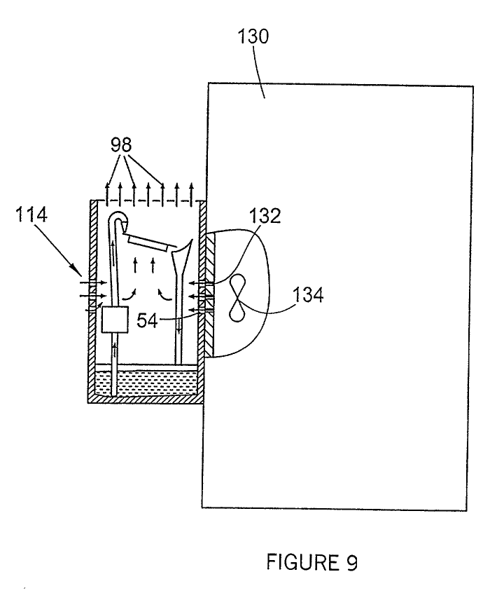

FIG. 9 shows a schematic view of a computer with a device for enriching air according to the invention.

The device according to the invention comprises a storage vessel 10 for receiving air treatment agent 12. In the Example shown, vessel 10 has a cylindrical shape. However, the storage vessel may also have a rectangular or other shape. A bottom 14 of the storage vessel 10 is inclined in the direction of an outlet 16 so that the air treatment agent 12 flows towards outlet 16, even when only a small amount of air treatment agent 12 is contained in storage vessel 10. The volume of storage vessel 10 is such that storage vessel 10 can be filled with a quantity of air treatment agent 12 which will last for about one month.

For filling storage vessel 10, it has a charging hole 18 which is connected to a cylindrical charging piece 20. The charging piece 20 is closed by a cover 22, which has a frustra-conical shape in the represented Example. For generating air inlet openings 24 which remain open even when the cover 22 is closed, the end of the cylindrical piece 20 facing towards the cover 22 is closed, the end of the cylindrical piece 20 facing towards the cover 22 is provided with crenelated portions or the like. The air inlet openings 24 ensure that there is always ambient pressure in the storage vessel 10, and the conveyed quantity of air treatment agent cannot be influenced by reduced pressure.

The outlet 16 is provided with an outlet piece 26 to which a pipe 30 is attached through a flange 28. To prevent dust or other particles from entering into the pipe 30, a fine-meshed filter, for example, may be provided in the outlet 16. To provide a filter in the region of the outlet 16 has the advantage that it can be readily changed and cleaned through the charging hole 18 which is arranged opposite the outlet 16. The filter has such a design that particles having a diameter of up to more than about 0.1 mm, preferably more than 0.02 mm, are filtered off.

To prevent dust or other particles from entering into the storage vessel 10, a corresponding particle filter may further be provided in the inlet openings 24.

The pipe 30 is connected with a mixing vessel 32, the end 34 of pipe 30 projecting into mixing vessel 32. In the represented Example, the pipe 30 is closed at its end 34 with a plate or disk 36, which is preferably exchangeable. Preferably in the center of disk 36, an outlet 38 in the form of a circular hole is provided as a dosing means. The hole has a diameter of preferably from 0.02 to 0.1 mm. This ensures that the above described small quantities of air treatment agent 12 reach the mixing vessel 32.

In pipe 30, a shut-off valve 40 with a handwheel 42 is provided for interrupting the supply of air treatment agent to mixing vessel 32. Thus, valve 40 exclusively serves for turning off the device. A corresponding valve could also be arranged immediately behind outlet 16 of storage vessel 10.

Further, a fine-meshed sieve 44 is provided within pipe 30, in addition to or instead of a fine-meshed sieve provided within outlet 16. The mesh of sieve 44 is preferably the same as that of the sieve optionally present within outlet 16. After closing the valve 40, sieve 44 can be exchanged so that sieve 44 can be readily cleaned.

Due to the different levels of storage vessel 10 and the outlet 38 of the closing means, a pump or the like for conveying the air treatment agent 12 is not necessary. The supply of air treatment agent 12 to the mixing vessel 32 is effected exclusively by gravity. The maximum difference of level between outlet 38 and a maximum filling height of storage vessel 10 is between 5 and 15 cm, especially between 5 and 10 cm. This ensures that the air treatment agent 12 has a pressure at the outlet 38 which is but slightly higher than the ambient pressure. Thus, the supply of air treatment agent 12 to mixing vessel 32 is quasi pressureless. In order to realize as low as possible a pressure difference between storage vessel 10 when completely filled and storage vessel 10 when almost empty, the maximum difference between maximum and minimum filling levels of air treatment agent 12 in storage vessel 10 is preferably less than 6 cm, especially less than 4 cm. The size of the vessel is selected to provide a volume of about 300 to 400 ml.

Since the supply of the air treatment agent 12 to mixing vessel 32 is effected exclusively by gravity, this preferred embodiment requires no control, especially no flow-control valve or the like.

Within the mixing vessel 32, an evaporating means 46 is arranged. In the represented embodiment, the evaporating means 46 constitutes the bottom of the mixing vessel 32 so that the entire bottom of mixing vessel 32 is designed as an evaporating means 46. The evaporating means 46 comprises an evaporation dish 48 consisting of a material with a high heat conductivity. The evaporation dish is preferably made of aluminum. On the bottom of the evaporation dish, preferably on the outside thereof, a heating film 50 is provided. The heating film 50 is connected to an energy source. Preferably, the heating film 50 is fed by 12 Volt energy source and has a power input of 10 to 15 Watt. The heating film preferably heats the receptor dish 48 to 40-70° C., especially 50-60° C. Thus, the air treatment agent leaving the outlet 38 evaporates relatively slowly in receptor dish 48 and rises towards an outlet 52 of the mixing vessel 32. Preferably, the receptor dish 48 is inclined, sloping form outlet 38, so that the air treatment agent supplied to the receptor dish 48 spreads over the heated bottom of the receptor dish 48 and uniformly evaporates.

In the lateral wall of the mixing vessel 32, which preferably has rotational symmetry, air inlet openings 54 are provided. The air inlet openings 54 are preferably distributed uniformly around the perimeter to ensure a uniform supply of ambient air into the mixing vessel 32. The air supply through the air inlet openings 54 into the mixing vessel 32 is realized by a means for generating a current of air, such as a fan 56. The fan 56 is provided opposite the evaporating means 46 on the outlet 52 of mixing vessel 32. The fan sucks air through the air inlet openings 54 in the direction of the arrows 58 into the interior of mixing vessel 32 and blown into the space to be treated in the directions of the arrows 60.

Especially due to the funnel-shaped design of mixing vessel l32, whose cross-section increases from evaporating means 46 towards the outlet 52, a stack effect is produced within the mixing vessel 32. In order not to impede the evaporation of the air treatment agent in the receptor dish 48, the air inlet openings 54 are arranged at a distance from the evaporating means 46, i.e., above the receptor dish 48. The distance of the air inlet openings 54 provided closest to the evaporating means 46 is preferably from 2 to 8 cm, especially from 4 to 6 cm.

In principle, the second preferred embodiment as shown in FIG. 3 corresponds to the embodiment shown in FIGS. 1 and 2. Therefore, identical or similar components of the device are indicated with the same reference symbols.

The essential difference between the two preferred embodiments is that the space required for mounting the second preferred embodiment (FIG. 3) is smaller. This is achieved by allowing mixing vessel 32 to project through a storage vessel 62. Therefore, storage vessel 62 has openings 68, 70 in a bottom 64 and in an opposite wall 66. The mixing vessel 32 is inserted through openings 68, 70 so that the fan 56 is arranged above the storage vessel 10.

The arrangement of storage vessel 62 with respect to mixing vessel 32 has the consequence that the outlet 16 of the mixing vessel is not arranged in the center, but on one side of the storage vessel. The charging hole 18, the charging piece 20 and the cover 22 are in turn essentially arranged opposite the outlet 16.

The storage vessel 62 preferably has a circular side wall 72 in accordance with the storage vessel 10.

In the represented Example, the mixing vessel 32 has a circular-cylindrical rather than a funnel-shaped design. However, it is also possible to provide a funnel-shaped mixing vessel 32 in this embodiment.

The embodiment shown in FIG. 4 is another preferred embodiment which, as compared to the preferred embodiments of the invention described in FIGS. 1 to 3, exhibits an essential difference in that the evaporating means 46 has an evaporation surface 74 which is inclined relative to a horizontal plane. Components similar to those of the embodiments of the invention described in FIGS. 4 to 6 are indicated with the same reference symbols. Especially with respect to the quantities supplied to evaporating means 46 and the size of the storage vessel 10, this embodiment preferably essentially corresponds to the above described embodiments.

The air treatment agent 12 is pumped from a storage vessel through pipes 78, 80 to evaporating means 46 using a pump 76. The air treatment agent emerging from a feeding nozzle 82 emerges from a slot-shaped outlet 84 of the feeding nozzle. The slot 84 essentially extends over the entire width of the evaporation surface 74, which is rectangular in the Example shown. The evaporation surface 74 may be a simple metal sheet, preferably made of aluminum or copper, i.e., of a material having a high heat conductivity. The heating of the evaporation surface 74 is effected by the heating film 50. While the air treatment agent flows from an upper zone 76 over the inclined evaporation surface 74, a major portion of the air treatment agent is evaporated due to the heat supplied by the heating film 50.

The excess air treatment agent is collected by a recirculating means 88 and recirculated to the storage vessel 10 through a pipe 90. The recirculating means 88 is preferably funnel-shaped and extends over the entire width of evaporation surface 74. The feeding nozzle 82 and the recirculating means 88 are thus arranged opposite each other, the feeding nozzle being arranged in the upper zone 86 of the evaporation surface 74, and the recirculating means 88 being arranged in a lower zone 92 of evaporation surface 74.

For enriching the air with an air treatment agent, the embodiment shown in FIGS. 4 to 6 also comprises a means for generating a current of air, such as a fan 56. In the embodiment shown, the fan 56 is arranged below the evaporating means 46. Thus, the air sucked through the air inlet openings 54 in a mixing vessel is guided by fan 56 in the direction of the arrows 96. Since the air flows into the lower side of the evaporating means, it is guided outward along the lower side and flows past the exterior surfaces, i.e., laterally past the evaporating means 46, upwards towards outlets 52 and flows into the space to be treated in the direction of the arrows 98.

In the Example shown, the mixing vessel 94 simultaneously serves as a housing for the pump 76, the fan 56 and the evaporating means 46. Further, the storage vessel 10 is arranged within the vessel 94 serving as a housing.

For filling the storage vessel 10, a charging piece 100 which can be closed with a cover 102 is provided.

FIG. 7 shows a refrigerating means 110 with an outer wall 112 to which a device 114 for enriching air according to the invention is attached. The device 114 may be, for example, the embodiment of the invention as described in FIGS. 1 to 6. Preferably, instead of a storage vessel 10 (e.g., FIGS. 1 and 4), a separate storage vessel 116 is provided which may be arranged, for example, on a top side 118 of the refrigerator. The storage vessel 116 is a storage vessel the volume of which is selected such that the air treatment agent preferably must be replenished only at intervals of about 6 and more preferably about 12 months.

The air exhausted by device 114 and enriched with air treatment agent passes in the direction of an arrow 120 through a duct 122 into an interior space 124 of the refrigerator 110.

In another preferred embodiment of the invention (FIG. 8), the device 114 for enriching air is arranged within the interior space 124 of the refrigerator 110. The device 114 is preferably one of the above described preferred embodiments of the device. The air containing the air treatment agent is exhausted immediately into the interior space 124 of the refrigerator in the direction of arrow 126.

According to the embodiments described in FIGS. 1 to 6, a storage vessel clan be provided inside the refrigerator 110. Preferably, however, a larger storage vessel 116 is provided on a top surface 118 of the refrigerator. The storage vessel 116 is connected to the device 114 through a connecting hose 128. Further, the device 114 can additionally comprise a separate storage vessel which has a smaller volume than that of the storage vessel 116, and an intermediate chamber or the like, for example, is automatically replenished from the larger storage vessel 116.

Thus, the device 116 serves for replenishing a storage chamber provided within the device 114.

Preferably, the device 114 is surrounded by an isolating housing in order to avoid heating of the refrigerator by providing the device 114 according to the invention within the refrigerating space 124.

In another preferred embodiment of the invention (FIG. 9), a device for enriching air is provided externally at a housing 130 of a computer. In the stated Example, a device according to FIG. 4 is provided. Of course, it is also possible to provide the embodiments represented in FIGS. 1 and 3 or other embodiments of the device according to the invention. One particular design of the embodiment represented in FIG. 9 consists in that air exhaust slots 132 of the computer housing 130 are provided on the level of the air inlet openings 54. Thus, the transport of the air provided with the air treatment agent is performed immediately by a fan 134 of the computer. Thus, a separate fan 56 can be dispensed with in the device 114. It is thus possible to arrange the device 114 within the computer housing.

The invention further relates to:

a method for sterilizing and/or deodorizing a refrigerating means, especially for sterilizing a refrigerator, comprising evaporation of an antimicrobial composition and/or an odor-masking composition within the refrigerating means using a device as defined above; and

a method for sterilizing a computer and the room where the computer is installed, comprising evaporation of an antimicrobial composition by means of a device as defined above provided in the region of the aeration system of the computer.

The mentioned antimicrobial compositions and the odor-masking composition are preferably the above defined compositions (X) and (Y).

In the sterilization method for refrigerating means, the antimicrobial composition is evaporated in an amount of from 0.0001 to 0.5 ml per hour depending on the volume of the refrigerator. The antimicrobial composition may also contain the above defined odor-masking compound whereby any unpleasant smell which may prevail in the refrigerator can be reduced.

In the methods mentioned, the antimicrobial composition is introduced in such a way that a concentration thereof in the ambient air of smaller than or equal to 100 ppb (parts per billion), preferably about 10 ppb, is ensured, which is ensured by introducing 0.01 ml per m³ of space per hour. The invention will be further illustrated by the following Examples.

### EXAMPLES

In the following Examples, HiQ® Caire (consisting of 1% by weight of tannin, 0.5% by weight of essential oil, the balance being propylene glycol) was employed as the antimicrobial composition.

### Example 1

PC test. The germs in a personal computer and in the ambient air are reduced towards zero by employing HiQ® Caire and introducing it with the device according to the invention within the PC tower in the exhausting zone of the fan (see FIG. 9) at an applied quantity of 0.01 ml.m³/h (HiQ® Caire concentration of ˜10 ppb). The experimental results are shown in Table 1.

TABLE 1

| | mold* germ number per m³ | | | | |
|---|---|---|---|---|---|
| sampling/volume (I) | 500 | 100 | 50 | 20 | 10 |
| prior to | 58 | n.e.** | n.e. | n.e. | n.e |
| contamination | | | | | |
| immediately after | n.e. | 47,400 | >500,000 | >500,000 | >500,000 |
| contamination | | | | | |
| after 1 day | 4 | 0 | 0 | n.e. | 0 |
| after 2 days | 2 | 0 | 0 | n.e. | 0 |
| after 3 days | 0 | 0 | 0 | n.e. | n.e |
| after 4 days | 0 | n.e. | n.e. | n.e. | n.e. |

*mold = Aspergillus and Penicillium
**n.e. = not examined.

### Example 2

Refrigerator test. Using an evaporating means according to the invention, 0.5 ml per h of HiQ Caire was introduced into the refrigerator from the exterior (see FIG. 7). This significantly reduced the germs on the foods in the refrigerator. The experimental results are represented in Table 2.

TABLE 2

| | rice (comparative test) | rice with HiQ ® |
|---|---|---|
| total germ count cfu/g | 70 | 70 |
| Day 3 of study | 100 | 100 |
| Day 6 of study | 1.3 × 10⁵ | <10 |
| Day 8 of study | 6.4 × 10⁵ | <10 |

1. A device for enriching air with an air treatment agent, comprising:
a storage vessel for receiving liquid air treatment agent;
an evaporating means for heating the air treatment agent, connected with the storage vessel; and
a recirculating means for recirculating excess air treatment agent from the evaporating means into the storage vessel;
wherein said evaporating means has an inclined evaporation surface over which the air treatment agent to be evaporated flows.

2. The device according to claim 1, characterized in that said evaporation surface has an inclination of from 10° to 30° relative to a horizontal plane.

3. The device according to claim 1, characterized in that said evaporation surface has conveying grooves running in the direction of flow.

4. The device according to claim 1, characterized in that said evaporating means has a feeding nozzle for uniformly distributing the air treatment agent on the evaporation surface.

5. The device according to claim 4, characterized in that said feeding nozzle essentially extends over the entire width of the evaporation surface.

6. The device according to claim 1, characterized in that said recirculating means has a collecting means.

7. The device according to claim 4, characterized in that said recirculating means and said feeding nozzle are arranged opposite to each other.

8. The device according to claim 1, characterized by a mixing vessel which is connected with said evaporating means and through which air is flowing, for admixing vaporized air treatment agent into the air.

9. The device according to claim 1, characterized by an agent for generating an air current, wherein said agent for generating an air current is arranged below the evaporating means, so that air flows laterally past the evaporating means.

10. A device for enriching air with an air treatment agent, comprising:
a storage vessel for receiving liquid air treatment agent;
an evaporating means for heating the air treatment agent, connected with the storage vessel;
a dosing means provided between said storage vessel and said evaporating means for the quantity-restricted supply of liquid air treatment agent to said evaporating means; and
a mixing vessel which is connected with said evaporating means and through which air is flowing, for admixing vaporized air treatment agent into the air.

11. The device according to claim 10, characterized in that said dosing means has an outlet whose cross-sectional area is smaller than or equal to 0.0078 mm².

12. The device according to claim 10, characterized in that the storage vessel and the evaporating means are at different levels so that the supply of air treatment agent is effected exclusively by gravity.

13. The device according to claim 11, characterized in that said dosing means comprises a conveying means for conveying said air treatment agent into said mixing vessel.

14. The device according to claim 1, characterized in that said evaporating means is arranged within said mixing vessel.

15. The device according to claim 1, characterized in that said mixing vessel has at least one air inlet opening, and a means for generating a current of air which sucks air in through the air inlet opening and exhausts a mixture of air and vaporized air treatment agent out through an outlet assigned to the mixing vessel.

16. The device according to claim 1, characterized in that said evaporating means is arranged in a bottom zone of the mixing vessel, and the outlet is arranged opposite the evaporating means, to produce a stack effect.

17. The device according to claim 14, characterized in that said air inlet opening is provided in a lateral wall of said mixing vessel above said evaporating means.

18. The device according to claim 1, characterized in that said mixing vessel has a funnel-shaped design in the direction of the outlet.

19. The device according to claim 1, characterized in that the mixture of air and vaporized air treatment agent exhausted through the outlet of the mixing vessel has a content of air treatment agent per cubic meter of air of from 0.00001 ml to 0.5 ml.

20. The device according to claim 15, characterized in that the cross-sectional area of the air inlet opening is greater than the cross-sectional area of the outlet.

21. The device according to claim 1, characterized in that said storage vessel has a pressure compensation means so that ambient pressure prevails in the storage vessel.

22. The device according to claim 21, characterized in that said pressure compensation means comprises a particle filter.

23. The device according to claim 1, characterized in that said evaporating means comprises a receptor dish made of a material having a good heat conductivity for receiving air treatment agent, and a heating means, especially a heating film, is connected with said receptor dish.

24. The device according to claim 1, characterized in that a fine-meshed sieve is incorporated prior to said dosing means.

25. The device according to claim 1, characterized in that the content of air treatment agent in the mixture of air and air treatment agent supplied to the space to be treated is no greater than 500 ppb.

26. The device according to claim 1, characterized in that an antimicrobial composition is used as said air treatment agent.

27. The device according to claim 26, characterized in that said antimicrobial composition contains one or more GRAS flavoring agents or their derivatives.

28. The device according to claim 1, characterized in that an odor-masking composition which contains at least one odor-masking component (A) selected from terpenes, corn starch, manganese salts, essential oils and polyvinyl pyrrolidone is used as said air treatment agent.

29. The device according to claim 28, characterized in that said odor-masking component at least contains polyvinyl pyrrolidone having a molecular weight of from 10,000 to 60,000.

30. The device according to claim 28, wherein said odor-masking composition further contains a functional flavor component (B).

31. The device according to claim 30, characterized in that said functional flavor component (B) contains at least one of the following substances:
hexyl butyrate, octyl acetate, isobutyl isobutyrate, cis-3-hexene-1-yl acetate, γ-decalactone, ethyl caproate, butyl acetate, ethyl benzoate, ethyl butyrate, hexyl acetate, methyl caproate, phenylethyl alcohol, citronellol, undecyl aldehyde, benzylphenyl acetate, cinnamyl alcohol, eugenol, benzyl acetate, linalool, cis-jasmone, acetylmethyl anthranilat, cis-3-hexene-1-ol, cis-3-hexene-1-yl salicylate, methyl benzoate, methyl salicylate, geranyl acetate, cis-3-hexene-1-yl acetate, Litsea cubeba, orange oil, phenylpropyl alcohol and phenylethyl acetate.

32. A refrigerating means, especially a household refrigerator, comprising a device for enriching air according to claim 1.

33. The refrigerating means according to claim 32, characterized in that said device for enriching air is provided at a housing of a refrigerating means and said device for enriching air is connected with the interior space of the refrigerating means through a duct.

34. The refrigerating means according to claim 32, characterized in that said device for enriching air is provided in the interior space of the refrigerating means.

35. A computer comprising a device for enriching air according to claim 1.

36. The computer according to claim 35, characterized in that said device for enriching air is provided in the region of a computer aeration means.

37. The computer according to claim 36, characterized in that the conveying of the air treatment agent is effected by said computer aeration means.

38. The computer according to claim 35, characterized in that the heat generated by the computer serves as said evaporating means.

39. A method for sterilizing and/or deodorizing a refrigerating means, especially for sterilizing a refrigerator, comprising evaporation of at least one of an antimicrobial composition and an odor-masking composition within the refrigerating means using a device as defined in claim 1.

40. A method for sterilizing a computer and the room where the computer is installed, comprising evaporation of an antimicrobial composition using a device as defined in claim 1 provided in the region of the aeration system of the computer.

41. The method according to claim 39, wherein said antimicrobial composition contains at least one GRAS flavoring agent or its derivatives.

42. The method according to claim 39, wherein said antimicrobial composition is evaporated in an amount of from 0.01 to 0.5 ml/h for sterilizing a refrigerator.

43. The method according to claim 39, wherein the concentration of the antimicrobial composition in the refrigerating means, the computer or the room where the computer is installed is no greater than 500 ppb.
